(19) United States
(12) Patent Application Publication
(10) Pub. No.: US 2007/0185371 A1
(43) Pub. Date: Aug. 9, 2007

(54) Magnetic device and method to prevent gastroesophageal reflux, fecal incontinence and urinary incontinence

(76) Inventor: Mauro Bortolotti, Bologna (IT)

Correspondence Address: BROWDY AND NEIMARK, P.L.L.C.;624 NINTH STREET, NW, SUITE 300, WASHINGTON, DC, 20001-5303, US

(21) Appl. No.: 11/508,907

(22) Filed: Aug. 24, 2006

(30) Foreign Application Priority Data
Feb. 8, 2006 (IT) BO2006A000071

(51) Int. Cl. A61F 2/00; A61F 2/02

(52) U.S. Cl. 600/29; 600/30

(57) ABSTRACT

The invention consists in a couple of magnets in form of small plaques of variable dimensions and magnetic force, covered by a bio-compatible material, that are positioned by means of a special endoluminal delivery device or by means of a surgical intervention, close to the sphincters that are to be reinforced, with the opposite polarities face to face, so that, attracting to each other, they close the visceral lumen, preventing the reflux or the incontinence.

This method may be applied to the lower esophageal sphincter in order to prevent gastroesophageal reflux, to the anal sphincter to prevent fecal incontinence, and to the urethral sphincter to prevent urinary incontinence.

### FIELD OF THE INVENTION

The present invention relates generally to the medical field of visceral prosthetics with the aim of strengthen the function of closure of weak visceral sphincters, including the gastroesophageal, anal and urethral sphincters, in order to prevent, respectively, the gastroesophageal reflux, and fecal and urinary incontinence.

### BRIEF SUMMARY OF THE INVENTION

The invention consists in a couple of magnets in form of small plaques covered by a bio-compatible material that is positioned by means of a special endoluminal delivery device or by means of a surgical intervention, close to the sphincters that are to be reinforced, with the opposite polarities face to face so that attracting to each other they close the visceral lumen preventing the reflux or the incontinence.

The magnets must have a force of attraction that creates a closure sufficient to prevent the passage in the wrong direction, but at the same time must allow the passage in the right direction. In addition, the force of attraction must be suitable not only for the kind of the sphincter to correct, but also for its degree of incompetence.

The main advantage of the magnetic closure of the sphincters lies in the fact that, not only it is more efficient, but also, when the magnets are detached, it leaves a passage, which allows an easy transit of the content, at variance with the endoscopic techniques at present available, which try to prevent the wrong flux of content by means of a more or less rigid narrowing of the lumen of the visceral canal, that, however, represents an obstacle for the flux of the content in the right direction.

### BACKGROUND OF THE INVENTION

The function of the sphincters consists in the regulation of the content flux in visceral canals by means of a tonic contraction of their musculature that surrounds as a ring the lumen of the visceral canal. The sphincters prevent in some cases the abnormal retrograde flux of content (reflux) and in other cases the abnormal anterograde flux (incontinence) when the sphincter musculature becomes weak due to a disease, surgical intervention of without an apparent cause (idiopathic forms), the tone of closure decreases more or less remarkably, up to a complete absence in some cases, and, consequently, the sphincter become unable to control the content flux (incompetent sphincter).

At the esophageal level there is the lower esophageal sphincter, also called cardial sphincter, that prevents the gastric content to go back into the esophageal lumen (gastroesophageal reflux), whereas at anal level there is the anal sphincter, that prevents the leakage of feces contained in the rectal ampulla (fecal incontinence) and at urinary bladder level there is the bladder sphincter, also called urethral sphincter, that prevents the leakage of urine (urinary incontinence).

Consequently one can make three embodiments of the present invention: one for the gastroesophageal reflux, one for the fecal incontinence and one for the urinary incontinence, which will be described afterward after having described the pathophysiologic problems and the solutions available at present for each dysfunction.

The gastroesophageal reflux is due to an inability of the lower esophageal sphincter, that is the principal factor of the antireflux barrier, to keep closed the communication between the stomach cavity and the esophageal lumen in order to avoid the passage in the esophageal lumen the acid gastric content, that may give rise to more or less severe lesions not only of the esophageal mucosa (such as esophagitis, Barrett metaplasia and esophageal adenocarcinoma, etc.), but also in some cases of the mucosa of the airways (laryngitis, laryngospasm, larynx adenocarcinoma, cough, asthma, etc). At the present time the treatment of GER is based on the administration of drugs, as proton pump inhibitors (PPI), which block the secretion of hydrochloric acid from the oxinitic cells of the stomach, so decreasing the most important lesive factor in the genesis of GER dependent lesions. However, this treatment should be chronic and, being rather expensive, both patients and health insurance companies tend to reduce as much as possible the dosage and the duration of drug administration with consequent frequent relapses of the disease. In addition, there are some patients who are “non responders” to PPIs and others in whom besides the acid reflux there is also a bile reflux that is also highly damaging and is scarcely corrigible with drugs, The only correction in these case is represented by methods for increasing LES tone and decreasing the occurrence of prolonged inappropriate relaxations, that are not induced by swallowing and are one of the most important mechanisms of GER even if the LES tone is normal. (Dodds et al. N. Engl J Med 1982; 307:151).

Unfortunately the drugs used for increasing the LES tone are scarcely effective and is necessary to strengthen the antireflux barrier with mechanical methods.

The most popular of these methods at present available is the Nissen fundoplication, a surgical intervention, which is carried out under general anesthesia. However, it is not risk free, it does not work correctly in all patients, it may give rise to undesirable effects and is not for life, because after a decade the 70% of patients is returned to take PPIs (Lundell et al. Dig Dis Sci 2004; 22:161).

The Angelchik prosthesis has been by now definitely abandoned, because of severe complications, whereas the recent endoscopic techniques based on a narrowing of the esophageal lumen, such as ENDOCINCH PLICATOR and GATEKEEPER or on the destruction of the nervous fibers relaxing LES (STRETTA) are unable to prevent GER and improve esophagitis and may expose the patient to undesirable effects or more or less severe complications (Fennerty, DDW, Chicago 2005). Consequently there is the necessity of strengthen mechanically the antireflux function of LES with a method simple, effective, easily applicable, not expensive, devoid of risks and with only slight collateral effects.

The fecal incontinence is due to a loss of tonic contraction of the anal sphincter that does not retain anymore the feces into the rectum giving rise to fecal incontinence: The anal sphincter is formed by a ring of smooth muscle fibers (internal sphincter) and by a ring of a striated muscle fibers (external sphincter), has a length of 2.5-5 cm and a tone of closure of about 50-80 mmHg.

The cause of decrease in tone of the sphincter may be due to a muscle alterations for a disease (myopathy) traumatic lesions, surgical interventions, botulinum toxin infiltrations, or to a spinal cord or nerve alterations, or to an unknown cause (idiopathic). In addition in patients with idiopathic fecal incontinence there are spontaneous relaxations of the sphincter not only during the night but also during the day, that facilitate the fecal loss (Kumar et al Brit J Surg 1989; 76:635). The treatment is based on drugs able to increase the fecal consistency (loperamide), biofeedback, sacral transanal electrical stimulation, narrowing of the anal canal by means of infiltration with bio-compatible materials (antologous fat, collagen, etc.) and anal plugs. All these methods relieve, but do not resolve the problem in the large majority of patients and sometimes are scarcely tolerated.

In some cases one can resort to surgical interventions such as sphincteroplasty, creation of a neosphincter with muscle gracilis transposition, and colostomy or ileostomy in the most severe cases.

The surgical interventions, however, show scarce long-term results and are associated with frequent complications (Boharnuca A. E. et al. Gastroenterology 2003, 124:1672).

In some selected cases the artificial anus may be used. This is a device that is implanted in the abdominal cavity and consists in an inflatable ring that surrounds the distal portion of the rectum and is inflated and deflated on request with a semi-automatic pump. In some patients it gives good results, but is rather expensive and frequently may give rise to more or less severe complications, which cause rejection of the device in 36.6% of cases (Wang W D et al. Dis Colon Rectum 2004; 45:1139).

So there is the necessity of a device for the fecal incontinence that is simple, effective, devoid severe collateral effects and is not too much expensive.

The urinary incontinence that may be corrected by the present device is that due to a decrease or loss of tone of the urethral sphincter, so that any stress increase of intra-abdominal pressure, due to cough, sneeze, bending etc. may cause urine loss. The urethral sphincter is represented by an internal ring of smooth muscle fibers and another external ring of striated muscle, that surround the proximal portion of the urethra and with their tonic contraction prevent the loss of urine.

The decrease or loss of the sphincter tone may be caused by organic or functional alterations of the muscle (surgical interventions, traumatic lesions, myopathies, drugs, pregnancy, delivery, menopause, etc) or by diseases of the innervation (spinal cord or spinal nerve lesions, etc).

The treatment of the urinary incontinence is based on drugs (estrogens, alfa-1-antagonists, etc.) transvaginal or transrectal electrical stimulation and biofeedback to strengthen the pelvic floor muscle, narrowing of the urethral lumen with periurethral infiltrations of biocompatible materials, as collagen, etc, surgical interventions, and myoplasty of the sphincter with gracilis muscle.

All these methods, however, improve the urinary incontinence in a percentage of patients below 50% and, mainly with the infiltrations, there are complications, even severe, as urethral obstruction, necrosis, allergies, embolism, particle migration, etc.

In some selected cases one can implant the artificial bladder sphincter that consists of an inflatable ring surrounding the urethra and connected to a semi-automatic pump that inflate or deflate the ring under patient control. This method is effective in controlling the urine loss, but it has various contraindications, may give rise to more or less severe complications, that may lead to the explant of the device in about 30% of patients (Petero et al, J Urol 2006; 175:605) and is rather expensive. Even for the urinary incontinence the existence of several methods of treatment indicates that the problem is not resolved and there is the necessity of a method simple, effective, not too expensive, and devoid of severe complications.

### BRIEF DESCRIPTION OF THE DRAWINGS

FIG. 1 represents a schematic section following a vertical frontal plane through the lower portion of the esophageal wall (2) showing in profile the couple of magnets (1) inserted face to face in submucosal position (9) close to the LES (8).

FIG. 1bis represents a schematic section following a vertical lateral plane through the lower esophageal sphincter (8) showing a front view of the magnets (1).

FIG. 2 shows a section at the level of the magnets (1) following a horizontal plane indicated by the broken line a-a of the FIGS. 1 and 1bis.

FIG. 3 is a schematic view of a longitudinal section of the endoluminal delivery probe with the channel for the deployment of the magnets (12) and the operative chamber (14) seen in profile.

FIG. 4 is a frontal view of the operative chamber (14) at the distal end of the endoluminal device.

FIG. 5 is a transverse section of the delivery probe with the deployment channel attached to the internal wall of the device.

FIG. 6 represents the sequence of operations for the preparation of the mucosa and deployment of magnets. In FIG. 6/A the mucosa of the distal esophagus is sucked onto the perforated wall of the operative chamber. In FIG. 6/B the needle injects some mL of saline solution to perform a blister in the submucosa. In FIG. 6/C the end of the catheter with a blunted bolt creates a pouch in the submucosa. In FIG. 6/D the magnet seen in profile is pushed into the pouch.

FIG. 7 represents a longitudinal section of the modified delivery probe with two deployment channels (12 and 21) and two operative chambers (14 and 24) to insert the magnets without rotation of the probe.

FIG. 8 is a transverse section of the modified delivery probe showing the two deployment channels (12 and 21).

FIG. 9 represents a schematic section following a vertical frontal plane of the recto-anal region and showing the couple of magnets in profile (1) inserted with opposite polarities face to face, on the right and the left of the submucosa (27) of the anal canal.

FIG. 10 represents a transverse section through the anal canal at the level of magnets following the broken line b-b of the FIG. 9.

FIG. 11 shows an antero-posterior vertical section along the urethral lumen (34) showing the magnets (1) inserted in the urethral submucosa (35) just below the bladder neck (33).

FIG. 12 shows a transverse section of the urethra following the broken line c-c with the magnets (1) positioned in the submucosa (35) between the mucosal layer (36) and the muscular layer (32).

FIG. 13 represents a longitudinal section of the urethra and bladder showing, on the left of the drawing, the thoracoscope with the modified lumen that is introduced parallel to the urethra and deploys in the submucosa of the proximal left portion of the urethra the first magnet (1) seen in profile, whereas, on the right of the drawing, the thoracoscope deploys the other magnet (1) on the other side of the urethra at the level of the first magnet.

### DETAILED DESCRIPTION OF THE THREE EMBODIMENTS

The invention consists in a couple of magnets in form of small plaques covered by a bio-compatible material that are positioned close to the incompetent sphincters with the opposite polarities face to face, so that attracting to each other they close the visceral lumen preventing the reflux or the incontinence. However the sphincters that can be corrected have different anatomical and functional characteristics, that must be taken into consideration, when one has to make the magnetic devices.

In the distal portion of the esophagus there is the lower esophageal sphincter (LES) that prevents the gastroesophageal reflux (GER), in the distal portion of the rectum there is the anal sphincter, that prevents the fecal incontinence, and in the proximal portion of the urethra there is the urethral sphincter, that prevents the urinary incontinence.

Consequently one can make three embodiments of the present invention: one for the gastroesophageal reflux, another for the fecal incontinence and another for the urinary incontinence.

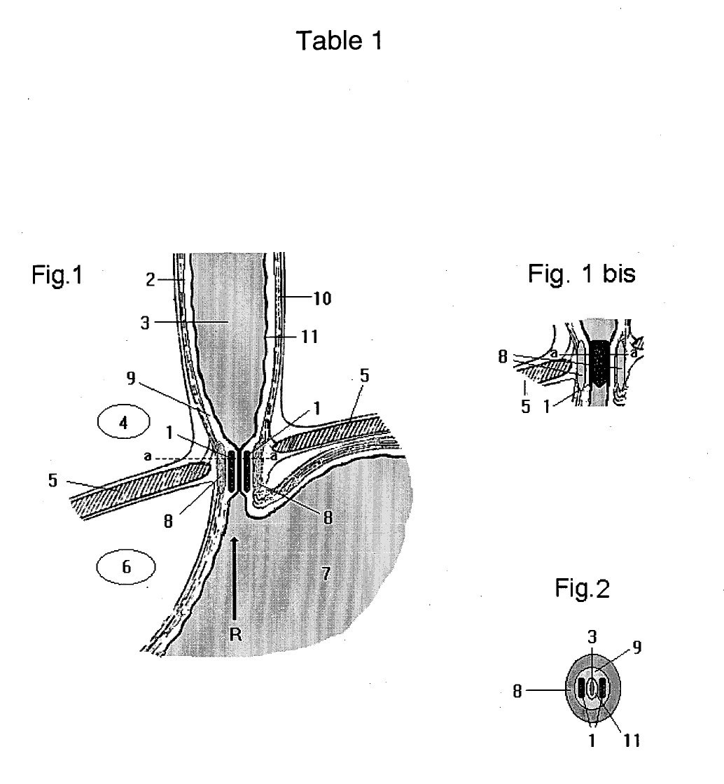

The 1^{st} embodiment of the invention for the gastroesophageal reflux (GER) is illustrated in FIG. 1, that represents a schematic section following a vertical frontal plane through the lower portion of the esophageal wall (2) at the level of the diaphragm (5) that separates from the abdominal cavity (6) the thoracic cavity (4). It consists in the insertion of a couple of magnets (1) in form of small plaques face to face in submucosal position (9) close to LES (8). In this manner they attract themselves compressing the mucosal layer (11), closing the lumen and preventing the reflux of gastric material (arrow R) from the gastric cavity (8), into the esophageal lumen (3). FIG. 1bis represents a schematic section following a vertical lateral plane through the lower esophageal sphincter (8) showing frontally the magnets (1). The FIG. 2 shows a section at the level of the magnets (1) and of LES (8) following a horizontal plane indicated by the broken line a-a of the FIGS. 1 and 1bis.

Alternatively the magnets may be inserted surgically in subserosal position close to the LES.

With regard to the characteristics of the two magnets, they can be made of “plastoferrite” or other magnetic material and must have when positioned a force of attraction sufficient to prevent the GER, but they must also allow the passage of the alimentary bolus from the esophagus to the stomach, without jeopardizing the circulation of the nutrient fluids in the surrounding tissues. Previous studies (Dodds et al. New Engl J Surg 1999; 4:405) demonstrated that a LES closure pressure of about 10 mmHg with length of 2 cm is sufficient to prevent GER. Consequently the magnets must close the esophageal lumen at the LES level with a pressure of at least 10 mmHg for about 2 cm in length. A bench experiment carried out with an artificial model of the esophagus demonstrated that it is possible to use magnets of 2 cm in length with a force of attraction that is overcome only by a pressure above 10 mmHg (Bortolotti M, J. Biomechanics 2006; 39:564). When the patient swallows a bolus, the magnets, that close the esophageal lumen at the LES level, are detached by the pressure that takes place in the esophageal lumen proximally to the magnets determined both by the peristaltic contraction, that normally is higher than 30 mmHg, a value markedly higher than the pressure of closure due to the magnets, and by the hydrostatic pressure due to the bolus descending into the esophagus, with the consequent opening of the passage between the esophagus and the stomach. Consequently the force of closure of magnets should not to be higher than about 30 mmHg to favor their detachment by the bolus arrival.

The force of attraction of the magnets must be chosen following the degree of LES impairment, which may range from the complete absence of LES tone (gastro-esophageal common cavity) to a normal tone with prolonged inappropriate relaxations, in order to obtain, when they are positioned, an intraluminal pressure that added to the residual pressure of the sphincter to strengthen, previously manometrically measured, an overall pressure of closure between 10 and 30 mmHg

This method offers important advantages in comparison with other endoscopic systems above mentioned (ENDOCINCH, PLICATOR and GATEKEEPER) that try to prevent GER narrowing more or less rigidly the esophageal lumen. In fact, on one hand, the couple of magnets closes completely the esophageal lumen, whereas the other endoscopic systems leave a more or less large passage through which the gastric juice leaches, on the other hand, when the bolus arrives, the magnets get away leaving a passage nearly large as the normal one, whereas with the other endoscopic systems the narrowed esophageal lumen widens with high difficulty without reaching anymore a near normal diameter and creating a more or less important obstacle to the passage of the bolus with consequent occurrence of dysphagia. In addition, at variance with the Nissen fundoplication, the magnetic closure allows both belching and vomiting without causing the “gas-bloat syndrome” due to a block of the air in the stomach consequent to fundoplication.

After the passage of the bolus in the stomach the magnets approach again, in consequence of their attracting force and of the pressure of surrounding tissues, closing again the communication between the stomach and esophagus.

The magnets must be covered by a layer or sleeve of biocompatibile material such as silicone or polyurethane foam, expanded polytetrafluorothylene or other materials that facilitate tissue ingrowth, such as extracellular matrix (surgisis), attached to the magnet surface in a variety of manners such as solvent bending, adhesives, etc. The surface of the magnets may have tissue retention structures to resist migration, such as hooks, barbs, tacks, clip, tissue adhesive or other system.

In addition the magnets can have holes connecting the two major surfaces with a diameter ranging from about 0.5 mm to about 3 mm and with an interhole distance ranging from about 1 mm to about 3 mm, with the aim of favour the circulation of nutrient fluids in the surrounding tissues that are compressed by the magnets.

The shape and dimensions of the magnets can also vary following the size of the patient and the site of positioning, that may be subserosal or submucosal, in the esophageal wall close to LES, with the main surface towards the esophageal lumen and the opposite polarities face to face (FIGS. 1 and 2). Anatomically the lower portion of the esophagus is made of a mucosal layer with a thickness of about 1-2 mm pluristratified epithelium, a submucosal layer with thickness of about 0.5-1 mm made of smooth, elastic and expansible connective tissue, and an inner circular muscular layer with a thickness of 2-3 mm somewhat stiffer and resistant, that gives rise to LES and is separated from the abdominal cavity by a serosal layer in the distal portion. In patients with GER there may be a sliding hiatal hernia, that, however, does not prevent the insertion of the magnets.

The magnets to be inserted in submucosal position, which is the preferred embodiment, may have a form of a rectangular small plaques with a thickness ranging from about 1 mm to about 2.5 mm, a width ranging from about 3 mm to about 6 mm and length ranging from about 20 mm to about 30 mm, with edges and corners rounded off except those facing the lumen to be closed and with a blunted bolt shape that may be easily inserted in the submucosal space.

The magnets to be surgically inserted in subserosal or intramuscular position may have the form of rectangular or ovoidal small plaques with edges and corners rounded off except those facing the lumen to be closed and with a thickness ranging from about 2 mm to about 4 mm, a width ranging from about 10 mm to about 20 mm and a length ranging from about 20 mm to about 40 mm. They must have a higher attraction force to obtain a pressure of 10-30 mmHg in the esophageal lumen, because they are more distant one from the other than those in submucosal position.

The insertion of the magnets in subserosal or intramuscular position may be accomplished by means of a laparoscopic or laparotomic surgical intervention, that creates two pouches of proper dimension on both sides of the esophageal wall close to the LES, so that the magnetic plaques can be positioned with the opposite polarities face to face, which can attract themselves closing the esophageal lumen.

The insertion of the magnets in the submucosal position close to the LES may be carried out in a variety of ways. An endoscope and proper tools may be utilized by a person skilled in the art to perform two pouches in the submucosa close to the LES one in front of the other, and to insert the couple of magnets in the two pouches, closing the mucosal openings by means of any variety of endoscopic closure techniques, such as conventional suture, ligating bands, clips, topical glue or adhesive patches.

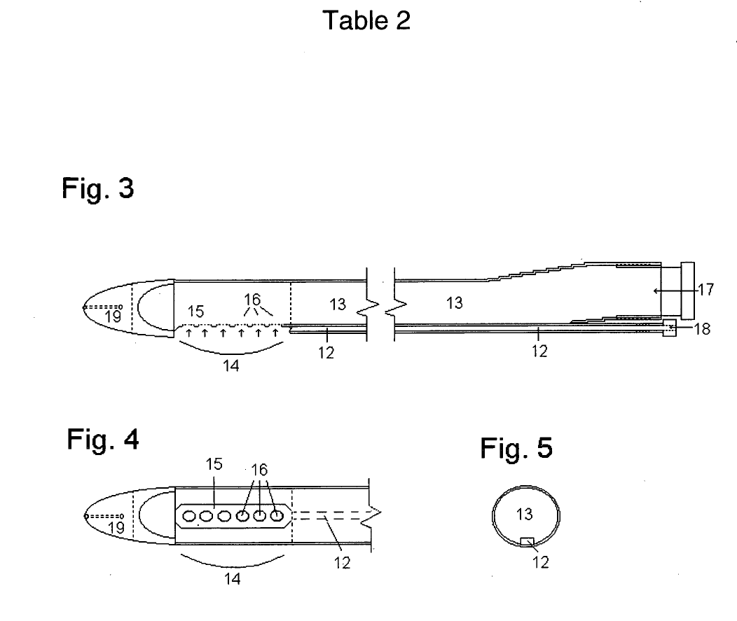

However the preferred method for submucosal magnets insertion is represented by the special endoluminal delivery probe illustrated in FIG. 3. This consists in a tube or catheter made of soft and flexible, transparent plastic material with a length variable from about 60 cm to 120 cm, preferably 90 cm, with an external diameter ranging from about 12 mm to about 18 mm and a side wall thickness of about 1 mm. The terminal portion of the tube is tapered with a guide wire lumen to facilitate placement in the esophagus (19).

The tube contains a channel for the deployment of magnets, called deployment channel (12), which is attached to the internal side of the wall and which has a lumen with a rectangular section with variable dimensions depending on the size of the magnets that are delivered: the longer side ranges from about 3.5 mm to about 11 mm and is tangent the circumference of the tube, whereas the shorter side ranges from about 1.5 mm to about 3 mm.

If the internal lumen of the delivery device that remains available has a diameter of about 11 mm, it may be used for the insertion of a standard endoscope (11) of 9 mm through a port with a seal (17), to control visually the deployment of magnets. The distal end of the deployment channel ends in a chamber (14) devoid of the external wall and with the internal wall (15) concave in shape and provided with holes (16) that connect the chamber with the main lumen of the tube. This chamber is of about 30 mm in length, about 5 mm in depth and about 7 mm in width and is called operative chamber (14). The proximal end of the deployment channel ends in a manifold that can be sealed and serve for the introduction (18) of the tools necessary for the preparation of the submucosal pouch where the magnets are deployed.

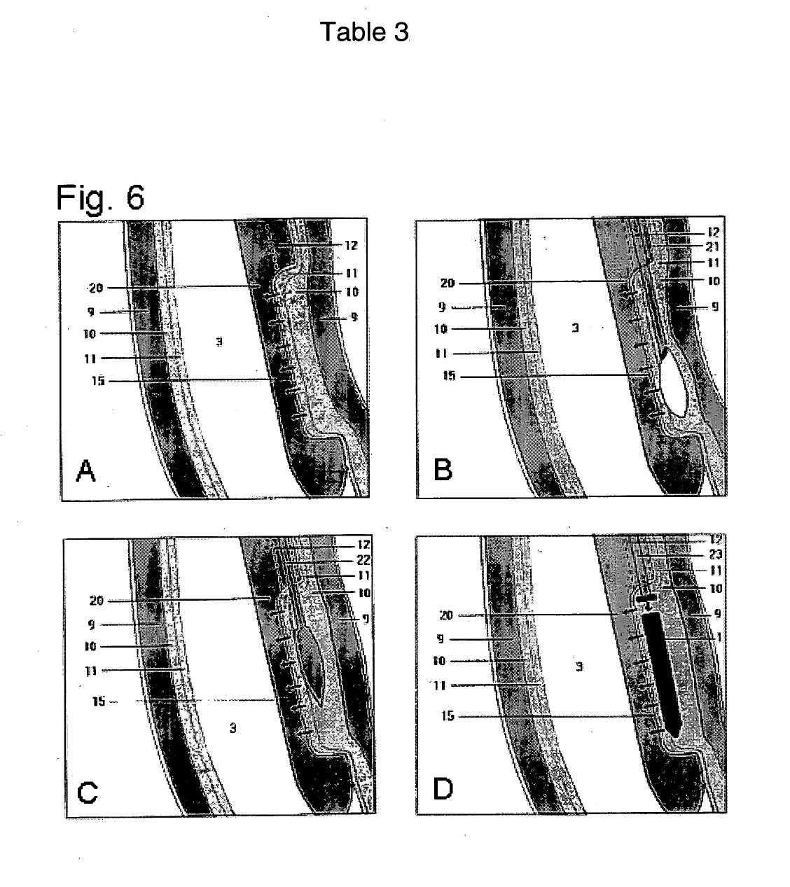

The operative chamber is positioned at the level of the distal esophageal mucosa, which is attracted against the internal perforated wall by a depression created by the aspirating system of the endoscope inserted in the main lumen. If the endoscope cannot be inserted, the depression is created by an external source attached to the proximal end of the main lumen of the delivery tube (17). In this manner the mucosa is immobilized during the operations for the preparation of the submucosal pouch and deployment of the magnet, that are illustrated in FIGS. 6A-D and described below.

After a proper local anesthesia and antibiotic covering, the delivery tube with or without the endoscope is introduced in the esophagus of the patient, then it is pushed following the guide wire, previously positioned in the stomach under endoscopic control, and the operative chamber is positioned in correspondence of the LES, the distance of which from the teeth was previously endoscopically measured. A radioscopic observation may be used to control the position of the operative chamber in absence of the endoscopic control. At this point the aspiration is activated with consequent depression in the main lumen of the tube, which attracts and immobilizes the mucosa against the perforated wall (15) of the operative chamber (14) (FIG. 6A). Then, by means of a catheter with a needle (21) introduced in the deployment channel (12), 3-6 mL of sterile normal saline solution are injected to create a blister in the submucosa with the aim of separating the mucosal layer from the muscular layer (FIG. 6B). At this point the first catheter is replaced by another catheter with the end in form of a blunt bolt (22), that is pushed distally for about 20-30 mm in the submucosal blister with the aim of creating a pouch in the submucosa (FIG. 6C). Alternatively, other systems may be used to create the submucosal pouch, such as electrocautery, laser or hydrodissection. Afterwards, the magnet (1) is pushed into the deployment channel by means of a catheter with a plat tip (23) and is deployed in the submucosal pouch (FIG. 6D) with one side facing the esophageal lumen, from which is separated by the mucosal layer, and the other side facing the LES muscular layer.

After the positioning of the magnet, the mucosal opening closes naturally or can be closed endoscopically with a variety of closing techniques, such as conventional suture, ligating bands, clips, topical glue or adhesive patches.

At this point the delivery tube is turned of 180° and the other magnet is inserted with the afore described procedure in the opposite wall of the esophagus at the same level of the other magnet, taking care of positioning towards the lumen the opposite polarity to that of the other magnet already deployed, so that the two magnets can attract themselves closing the lumen.

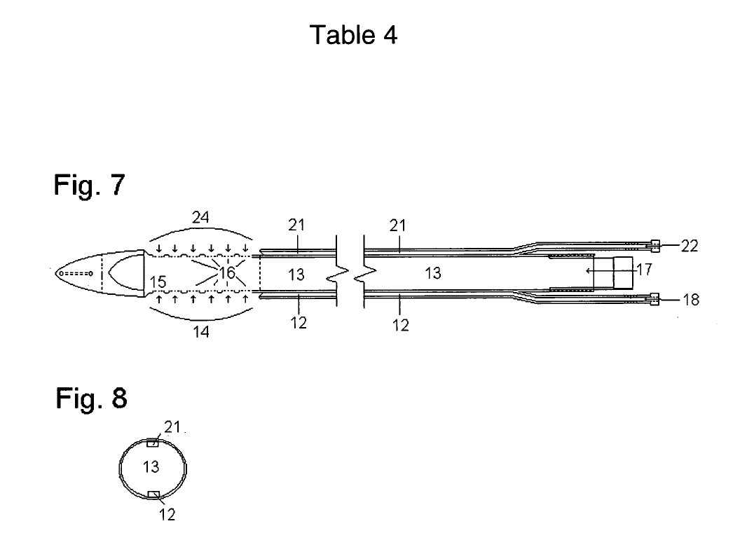

Alternatively the delivery tube described in FIG. 4 may be modified with the addition of another deployment channel (21) identical to the other, but positioned at 180°with respect to the other, and ending in another operative chamber (24) identical to the other (FIGS. 7 and 8). In this manner is not anymore necessary rotate the delivery tube in order to insert the second magnet. Once positioned the first magnet, it is sufficient to close hermetically the deployment channel (12), and continue in the new channel (21) the same procedure afore-described for the preparations of the submucosa and deployment of the second magnet (FIG. 6A-D).

In this embodiment, however, the delivery tube will have an internal lumen smaller than the other delivery tube of FIG. 2 that could allow in some cases the insertion of small endoscopes of less than 7 mm in diameter or no endoscopes. However, with this embodiment of the delivery tube one has the certainty that the two magnets are inserted exactly face to face to perfectly attract themselves.

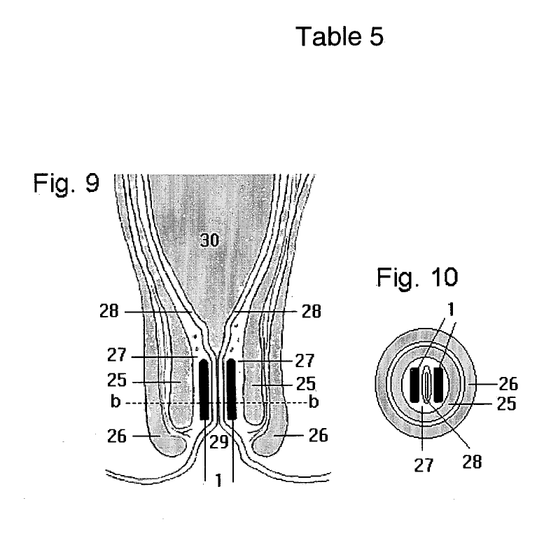

The 2^{nd} embodiment of the invention for the anal sphincter to prevent the fecal incontinence is illustrated in FIG. 9 that shows in a schematic section following a vertical frontal plane of the recto-anal region. It consists in the insertion of a couple of magnets (1) with the opposite polarities face to face, on the right and the left of the anal canal, in the submucosa (27), that is between the mucosa (28) and the musculature of the anal sphincters. The FIG. 10 represents a transversal section through the anal canal at the level of magnets following the broken line b-b of the FIG. 9.

Alternatively the magnets can be inserted between the internal (25) and external (26) anal sphincter.

In this manner the magnets attracting themselves close the anal canal preventing the loss of feces contained in the lumen of the rectal ampulla (30).

The magnets can be made of “plastoferrite” or other magnetic material and must have, when positioned a force of attraction variable ranging from about 10 mm to about 50 mmHg, that added to the more or less residual pressure of the anal sphincter to strengthen, previously manometrically measured, that may vary from a complete absence to a slight decrease, must create at the level of the lumen of the anal canal an overall pressure of closure of at least 50 mmHg sufficient to prevent the loss of feces, but not too high, because it must be overcome by the pressure that takes place in the rectal ampulla during defecation.

The magnets may have the form of rectangular or ovoidal small plaques with edges and corners rounded off except those facing the lumen to be closed.

The two magnets must be covered by a layer or sleeve of bio-compatible material, such as silicone or polyurethane foam, expanded polytetrafluorothylene or other material that facilitate tissue ingrowth, such as extracellular matrix (surgisis), that may be secured to the magnet surface in any variety of manners, such as by solvent bending, adhesive etc. The surface of the magnets may have tissue retention structures to resist migration such as hooks, barbs, tacks, clip, tissue adhesive or other system.

In addition, the magnets can have holes connecting the two major surfaces with a diameter ranging from about 0.5 mm to about 3 mm and with an interhole distance ranging from about 1 mm to about 3 mm, with the aim of favour the circulation of nutrient fluids in the surrounding tissues that are compressed by the magnets.

The shape and dimensions of the magnets may also vary depending on the size of the patient and the site of positioning, submucosal or between the internal and external anal sphincters.

The magnets can have a width ranging from about 10 mm to about 30 mm, a thickness ranging from about 1 mm to about 3.5 mm and length ranging from about 20 mm to about 40 mm.

The anal canal is 30-40 mm in length and is surrounded from the interior to the exterior, by a mucosal layer (28), by a submucosal layer (27), proximally made of smooth connective tissue, and by smooth muscle circular bundles of the internal anal sphincter (25) with a width of 4-6 mm. The latter is surrounded by the bundles of the striated musculature of the external anal sphincter (26).

The magnets should be positioned in the distal 3-4 cm of the anal canal with the major axis in vertical direction in the submucosal layer close to the anal sphincter or between the internal and external anal sphincters by means of a surgical intervention. After local or spinal anesthesia and an antibiotic covering, a retractor is inserted in the anal canal to expose the last portion of the anal canal on the right or left of the anus.

Then, if one chose to insert the magnets in the submucosa, the latter is infiltrated with a sterile normal saline solution from the more distal portion of the anal canal, above the “alba” line, to obtain a blister of about 3-4 cm in length and about 2-3 cm in width that separates the mucosa layer from the muscular layer. Then an incision of the mucosa above the “alba” line is made on one side of the anal canal and by means of a blunted scissors or other proper surgical tools or by means of the inflation of an ovoidal balloon with a length of about 3-4 cm and a diameter of about 2-3 cm the mucosal layer is separated from the muscular layer to create a pouch of sufficient dimensions. Alternatively other methods may be used to create the pouch, such as laser, electro-cauthery or hydrodissection. Once create the pouch the magnet is inserted and the incision is closed with one or two suture points or other proper systems, such as clips, glue, adhesive patches, etc.

Once completed the positioning of one magnet on one side of the anal canal, the same operation is carried out in the other side, taking cure of inserting the magnets with the opposite polarities face to face so that they can attract themselves closing the anal canal.

Alternatively the pouches for the magnets may be created between the internal and external anal sphincter muscular bundles, especially in patients with hemorrhoids, that may create some difficulty for the submucosal insertion of magnets. In this case, however the magnets must have a bigger dimension and a higher attraction force, because the distance between them is increased.

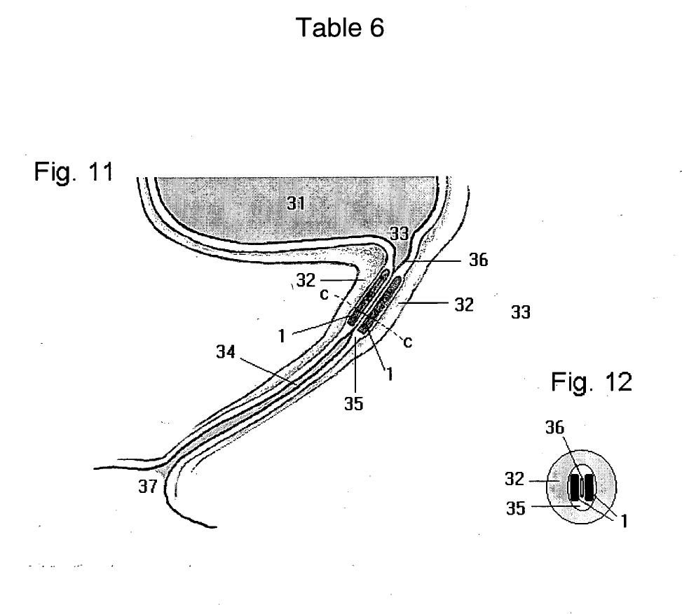

The 3^{rd} embodiment for the urethral sphincter to prevent the urinary incontinence is illustrated in the FIG. 11 that shows a longitudinal anteroposterior section along the urethral lumen (34). It consists in the insertion of a couple of magnet in the urethral submucosa (35) between the mucosa layer (36) and the muscular layer of the urethral sphincter (32), just below the bladder neck (33), with the opposite polarities face to face that reciprocally attract closing the urethral lumen with a pressure sufficient to prevent a loss of the urine contained in the bladder lumen (31).

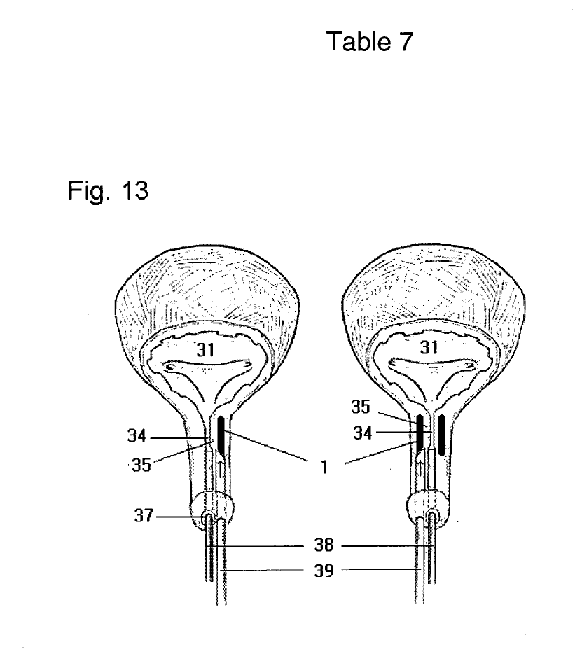

The magnets can be made of “plastoferrite” or other magnetic material and must have, when positioned, a force of attraction variable from about 20 mmHg to about 60 mmHg that added to the more or less residual pressure of the urethral sphincter to strengthen, previously manometrically measured, must create at the level of the lumen of the urethral canal an overall pressure of closure at least 60 mmHg, sufficient to prevent the loss of urine, but not too much higher, because it must to be overcome by the pressure that takes place in the urinary bladder during urination. The FIG. 13 shows a transverse section of the urethra following the broken line c-c with the magnets (1) positioned in the submucosa (35) between the mucosal layer (36) and the muscular layer (32).

The two magnets must be covered by a layer or sleeve of bio-compatible material, such as silicone or polyurethane foam, expanded polytetrafluorothylene or other materials that facilitate tissue ingrowth, such as extracellular matrix (surgisis), and may be secured to the magnet surface in any variety of manners, such as solvent bending, adhesive, etc. The surface of the magnets may have tissue retention structures to resist migration such as hooks, barbs, tacks, clip, tissue adhesive or other system.

In addition the magnets can have holes connecting the two major surfaces that toward the mucosa the opposite one with a diameter ranging from about 0.5 mm to about 3 mm and with an interhole distance ranging from about 1 mm to about 3 mm, with the aim of favour the circulation of nutrient fluids in the surrounding tissues that are compressed by the magnets.

The magnets may have the form of rectangular or ovoidal small plaques with edges and corners rounded off except those facing the lumen to be closed.

The shape and dimensions of the magnets may vary depending on the size of the patient and the site of positioning, submucosal or between the smooth and striated sphincters.

The magnets can have a width ranging from about 1.5 mm to about 3.5 mm, a thickness ranging from about 1 mm to about 3 mm and a length ranging from about 10 mm to about 25 mm.

This application is more easily performed in the women because the female urethra, at variance with the male one, is almost rectilinear and shorter. In fact it has a length of about 30-40 mm, a diameter of about 7-8 mm with a wall width of about 5-6 mm, that, being a lot elastic, can be dilated up to a diameter of about 20 mm without problems. The wall is formed, from the interior to the exterior, by a mucosal layer with small longitudinal folds that disappear with distension of the lumen, by a large submucosal layer of smooth connective rich in elastic fibers and by a muscular layer of the urethral sphincter represented by circular and longitudinal smooth muscle bundles, that extend to external urethral orifice (37). The median portion of the urethra is surrounded by circular bundles of striated musculature (37) that make up the striated urethral sphincter under voluntary control.

The insertion of magnets can be performed in three manners: through the urethra by means of an operative cystoscope, or by means of a special endoluminal delivery device, or through the periurethral tissues by means of a modified thoracoscope.

The first way of magnet application is performed with a cystoscope with a diameter sufficiently large that allows to insert the magnets in the submucosa of the proximal portion of the urethral canal, first on one side and then on the other side. After a proper antibiotic covering and local anesthetic preparation, the cystoscope is inserted up to the proximal portion of the urethra, just below the bladder neck, and then some mL of sterile normal saline solution are infiltrated in the submucosa to create a blister of about 2-3 cm in length

Then the mucosa of the lower portion of the blister is cut and by means of a blunted tool a pouch in the submucosa is created, which may be enlarged to a size sufficient for the magnet introduction by inflating a small balloon of about 2-3 mm in diameter and 10-15 mm in length. After positioning the magnet, the mucosal opening closes naturally or can be closed endoscopically with a variety of closing techniques, such as conventional suture, ligating bands, clips, topical glue or adhesive patches.

Afterwards, the other magnet is inserted in the submucosa with the same procedure at the same level of the urethral canal, but at 180° with respect to the first one, taking care of keep the opposite polarities face to face so that the magnets can attract themselves closing the urethral lumen.

In alternative to the magnet insertion with the cystoscope, one may utilize an endourethral delivery probe similar to that used for the esophagus illustrated in FIGS. 3.4 and 5, but of smaller dimensions. The delivery probe is made of flexible, smooth and transparent plastic material of about 25 cm in length, with a diameter of about 8 mm carrying a channel for the deployment of the magnets (12) with a rectangular section, having the main side tangent to the circumference with a length ranging from about 2.5 mm to about 4.0 mm and the other side with a length ranging from about 1 mm to about 2.5 mm.

If the deployment channel is small, the main lumen of the delivery tube (13) could allow the insertion of a flexible cystoscope or ureteroscope of small diameter to control the operations for magnet positioning and to create inside the main lumen of the delivery probe a depression necessary for fixing the mucosa to the perforated wall (15) of the operative chamber (14). If the main lumen does not allow the introduction of an endoscope the depression is given by an external source attached to the proximal end of the delivery tube (17). In this case a “blind” insertion of the magnets can be performed after a previous accurate endoscopic measure of the level where the magnets are to be inserted. The operative chamber (14), where ends the delivery channel, may have a length ranging from about 10 mm to about 25 mm, a depth ranging from about 3 mm to about 4 mm and a width ranging from about 3 mm to about 5 mm.

After having done a local anesthesia and antibiotic covering, the delivery probe is introduced in the urethra through the external orifice and the operative chamber is positioned in the proximal portion of the urethra, performing all the operations for positioning the magnet described in FIG. 6 A-D.

After the insertion of the magnet, the mucosal opening closes naturally or can be closed endoscopically with a variety of closing techniques, such as conventional suture, ligating bands, clips, topical glue or adhesive patches.

Then the endourethral catheter is turned of 180° and the other magnet is inserted at the same level with the same procedure, taking care of keep the opposite polarities face to face, so that the magnets can attract themselves closing the urethral lumen.

Alternatively one may use another endourethral delivery probe, similar to that used for the esophagus illustrated in FIGS. 7 and 8, with two deployment channels (12 and 21) and two operative chambers (14 and 24), one in front of the other at 180°, so that it is not necessary to rotate the catheter of 180° to insert the second magnet and one has the certainty that the two magnets are inserted one in front of the other to attract themselves in a perfect manner.

The third manner for magnet insertion consist in the use of a modified thoracoscope which has the advantage of leaving intact the mucosa, as it is passed through the periurethral tissues, close to the urethra, up to its proximal portion. First of all, with the patient under general or spinal anesthesia and antibiotic covering, a needle is inserted on the left of the urinary meatus and pushed proximally to infiltrate the periurethral tissues up to the proximal portion of the urethra with some mL of sterile normal saline solution in order to separate the mucosal layer from the muscular layer or, alternatively, the smooth sphincter layers from the striated sphincter layers. Then a small thoracoscope of about 8 mm in external diameter carrying a deployment channel with a rectangular section of the lumen having the major side tangential to the urethral wall with a length ranging from about 2.5 mm to about 4.0 mm and the other side ranging from about 1.5 mm to about 3.5 mm. To visually control the operations of magnet positioning a flexible cystoscope may be inserted in the lumen of the thoracoscope, if possible. The thoracoscope is pushed through the periurethral tissues to reach the submucosa or the boundary between the smooth and striated sphincters, with the technique known by the skilled persons in similar cases. Then by using the proper tools, such as blunted catheters and inflatable mini-balloons of 2-3 mm in diameter, inserted through the lumen of thoracoscope, a pouch is created for the magnet and the magnet is pushed through deployment channel in the submucosal or intramuscular pouch. Finally the thoracoscope is slowly taken off, whereas the magnet remains in the periurethral pouch. One can perform an ultrasonographic examination to control whether the magnet is in correct position.

Then the same procedure is carried out for the insertion of the second magnet in the opposite side at 180°, at the same level of the first magnet, taking care of keep the opposite polarities face to face, so that they can attract perfectly and close the urethral lumen.

1. Method and device to strengthen the insufficient function of closure of the visceral sphincters, such as lower esophageal sphincter, anal sphincter and urethral sphincter with the aim to prevent, respectively, the gastroesophageal reflux, the fecal incontinence and the urinary incontinence by means of a couple of identical magnets inserted surgically of by means special endoluminal delivery devices, close to the visceral sphincter with the opposite polarities face to face, so that attracting themselves, close the lumen of the visceral canal.

2. Device as in claim 1, characterized in that the magnets are made of “plastoferrite” or other magnetic material.

3. Device as in claim 1, characterized in that the magnets of each couple have the form of small plaques equal and symmetric with the faces showing opposite polarities.

4. Device as in claim 1, characterized in that the magnets have holes connecting the two main surfaces with a diameter ranging from about 0.5 mm to about 3 mm at intervals ranging from about 1 mm to about 3 mm.

5. Device as in claim 1, characterized in that the magnet surface have hooks, barbs, tacks, clips, tissue adhesive and other system to resist migration in the neighboring tissues.

6. Device as in claim 1 characterized in that the magnets are covered by a biocompatible or biologically inert material such as silicone, polyurethane foam, expanded PTFE or other material that facilitates the cellular ingrowth, such as extracellular matrix (surgisis).

7. Device as in claim 1, characterized in that the magnets to be inserted in the esophageal submucosa close to the lower esophageal sphincter have the form of small plaques ending in form of a blunt bolt and have edges and corners rounded off except those facing the esophageal lumen.

8. Devices as in claim 7, characterized in that the magnets to be inserted in the esophageal submucosa have a thickness ranging from about 1 mm to about 2.5 mm, a width ranging from about 3 mm to about 10 mm and a length ranging from about 20 mm to about 30 mm.

9. Device as in claim 7, characterized in that the magnets, once inserted in the esophageal submucosa, must show a force of attraction between themselves, that, added to the residual pressure of the hypotonic sphincter, previously manometrically measured, must give rise to a pressure ranging from about 10 mmHg to about 30 mmHg in the esophageal lumen.

10. Device as in claim 7, characterized in that the shape and dimensions of the submucosal magnets are variable to be adapted to the different patient built, to the different site of positioning and to the different force of attraction of the magnetic materials.

11. Device as in claim 1, characterized in that the magnets to be surgically positioned in subserosal or intramuscular position close to the lower esophageal sphincter have a form of small ovoidal or rectangular plaque with rounded off edges and corners, except those facing the esophageal lumen.

12. Device as in claim 11 characterized in that the magnets to be inserted surgically in the esophageal wall have a thickness ranging from about 2 mm to about 4 mm to a width ranging from about 10 mm to about 20 mm and a length ranging from about 20 mm to about 30 mm.

13. Device as in claim 11, characterized in that the force of attraction of subserosal or intramuscular magnets once positioned determines, in addition to the residual sphincter pressure previously manometrically measured, a pressure ranging from about 10 mmHg to about 30 mmHg in the esophageal lumen.

14. Device as in claim 11, characterized in that the shape and dimensions of the subserosal or intramuscular magnets are variable to be adapted to the different patients built and to the different magnetic materials with different force of attraction.

15. Device and method as in claim 1, characterized in that the endoluminal device for the delivery of the magnets in the esophageal submucosa is represented by a tube made of soft, flexible and transparent plastic material with a length from about 60 cm to about 120 cm, preferably 90 cm, and an external diameter ranging from about 12 mm to about 18 mm, with a side wall thickness of about 1 mm containing a channel for the deployment of magnets with a rectangular lumen having a shorter side ranging from about 1.5 mm to about 3 mm and a larger side ranging from about 3.5 mm to about 11 mm that ends and an operative chamber with a length of about 30 mm a width of about 30 mm and a depth of about 5 mm, devoid an external wall and with the internal wall perforated and communicating with the main lumen of the tube, where a depression is applied to block the mucosa against the perforated wall to perform the operations for the magnet deployment with proper tools.

16. Device and method as in claim 15, characterized in that the device for the delivery of the magnets in the esophageal submucosa close to the lower esophageal sphincter is provided with a couple of deployment channels at 180° one from the other, each of them similar in shape and dimensions to that described in claim 15 and ending in two separate operative chambers at 180° one from the other, similar in shape and dimension to that described in claim 15, that serve to position the magnets one in front of the other in the esophageal submucosa, avoiding the necessity of rotating the delivery device.

17. Device as in claim 1, characterized in that the magnets intended to be surgically inserted in the wall of the anal canal for treating the fecal incontinence have the form of a small ovoidal or rectangular plaque with corners and edges rounded off, except those facing the esophageal lumen.

18. Device as in claim 17, characterized in that the shape and dimensions of the submucosal magnets are variable to be adapted to the different patients built, to the different site of positioning and to the different force of attraction of the magnetic materials.

19. Device as in claim 17, characterized in that the anal magnets have a width ranging from about 20 mm to about 30 mm thickness ranging from about 1 mm to about 3.5 mm and a length ranging from about 20 mm to about 40 mm that is added to that of the anal sphincter.

20. Device as in claim 17, characterized in that the attraction force of the anal magnets induces after implantation a pressure ranging from about 10 mmHg to about 50 mmHg in the lumen of the anal canal that is added to the residual pressure of the incompetent anal sphincter previously manometrically measured that determines an overall pressure of closure of at least 50 mmHg sufficient to prevent the loss of feces.

21. Device and method as in claim 1, characterized in that the magnets intended to be inserted endoscopically or surgically in the proximal portion of the urethral wall to prevent the urinary incontinence have the form of a small plaque ending with a blunted bolt and with edges and corners rounded off, except those facing the esophageal lumen.

22. Device as in claim 21, characterized in that the shape and dimensions of the submucosal magnets are variable to be adapted to the different patients built, to the different site of positioning and to the different force of attraction of the magnetic materials.

23. Device as in claim 21, characterized in that the urethral magnets have a width ranging from about 1.5 to about 3.5 mm, a thickness ranging from about 1 mm to about 3 mm and a length ranging from about 10 mm to about 25 mm.

24. Device as in claim 21, characterized in that the attraction force of urethral magnets induces after implantation a pressure ranging from about 20 mmHg to about 60 mmHg in the urethral lumen, that is added to the residual pressure of the incompetent urethral sphincter previously manometrically measured determines an overall pressure of closure of at least 60 mmHg sufficient to prevent the loss of urine.

25. Device and method as in claim 21, characterized in that the device for the delivery of magnets at the level of urethral submucosa is represented by a tube made of soft and flexible transparent plastic material and is similar to that used for the esophageal magnets delivery, but with a length ranging from about 20 cm to about 40 cm, external diameter of about 8 mm and containing a deployment channel with a rectangular lumen having the shorter side ranging from about 1.5 to about 2.5 mm and the longer side tangent to the circumference ranging from about 2.5 mm to about 4 mm, that ends in an operative chamber with a length ranging from about 15 mm to about 25 mm, a depth ranging from about 3 mm to about 5 mm, devoid of an external wall and with the internal wall perforated and communicating with the main lumen of the delivery tube, where a depression is applied to block the mucosa against the perforated wall to perform the operations for the magnets deployment with proper tools.

26. Device as in claim 25, characterized in that the device for delivery of magnets in the esophageal submucosa close to the lower esophageal sphincter is provided with a couple of deployment channels at 180° one from the other, each similar in shape and dimensions to that described in claim 15, ending in two separate operative chambers at 180° one from the other, similar in shape and dimensions to that described in claim 25, that serve to position the magnets one in front of the other in the esophageal submucosa, avoiding the necessity of rotating the delivery device.

27. Device as in claim 1, characterized in that the urethral magnets are inserted in the submucosa or between the two urethral sphincters through the periurethral tissues by means of a modified thoracoscope of about 8 mm in external diameter, carrying a deployment channel with a rectangular section of the lumen having the main side tangential to the urethral wall ranging from about 2.5 mm to about 4.0 mm and the other ranging from about 1.5 to about 3 mm.
